(19) United States
(12) Patent Application Publication
(10) Pub. No.: US 2013/0023010 A1
(43) Pub. Date: Jan. 24, 2013

(54) METHOD FOR REVERSE TRANSCRIPTION POLYMERASE CHAIN REACTION

(71) Applicants: Ping Hua Teng, Taichung City (TW); Cheng Su, Taichung City (TW); Sih Ying Wu, Taichung City (TW); Fu Chun Li, Taichung City (TW)

(72) Inventors: Ping Hua Teng, Taichung City (TW); Cheng Su, Taichung City (TW); Sih Ying Wu, Taichung City (TW); Fu Chun Li, Taichung City (TW)

(73) Assignee: GENEREACH BIOTECHNOLOGY CORP., Taichung City (TW)

(21) Appl. No.: 13/377,490

(22) PCT Filed: Jul. 21, 2011
(86) PCT No.: PCT/CN11/77449

§ 371 Date: Dec. 9, 2011

(51) Int. Cl. C12P 19/34

(52) U.S. Cl. 435/91.2

(57) ABSTRACT

A method for reverse transcription polymerase chain reaction comprises steps: preparing a capillary, and adding a reverse transcription enzyme into the capillary; and performing a lyophilization process on the RT enzyme contained by the capillary to fabricate the RT enzyme into a lyophilized RT reagent in the capillary. Therefore, a buffer solution, an RNA sample, a polymerase and a primer solution can be added into the capillary to re-dissolve the lyophilized RT reagent and enable a reverse transcription reaction and a polymerase chain reaction of the RNA sample to directly take place inside the capillary, so as to promote convenience and efficiency of experiment.

### FIELD OF THE INVENTION

The present invention relates to a biochemical reaction method, particularly to a method for reverse transcription polymerase chain reaction, which enables a reverse transcription reaction and a polymerase chain reaction to take place in an identical device, whereby to promote convenience of experimental operations.

### BACKGROUND OF THE INVENTION

RT-PCR (Reverse Transcription Polymerase Chain Reaction) is used to detect RNA. In RT-PCR, an mRNA (message RNA) is added to an RT (Reverse Transcription) reagent to generate cDNA (complementary DNA). Then, the DNA target sequences are amplified million times by PCR (Polymerase Chain Reaction) so as to facilitate analysis.

In a general RT-PCR process, firstly an RT reagent is added to a test tube; next a sample is added to the test tube to undertake an RT reaction; next the cDNA generated by the RT reaction is added to a PCR tube; then the enzyme and buffer solution required by PCR are added to the PCR tube to enable PCR. In the conventional technology, the sample and reaction product are respectively added to different test tubes in different batches. Such a process exposes the sample and product to the air for a longer time and is more likely to contaminate the sample and product, which may cause false positive results.

Besides, the reaction product needs to be stored at low temperature to maintain the stabilization of the product and the activity of the enzyme. The low-temperature process inconveniences transportation and storage. Further, the low-temperature storage device is expensive. Furthermore, if inappropriately stored, the product may denature, and the enzyme may lose activity, which may affect the accuracy of the test result.

### SUMMARY OF THE INVENTION

The primary objective of the present invention is to provide an RT-PCR method, wherein an enzyme required by an RT reaction is fabricated into a lyophilized RT reagent in a capillary, and wherein an RNA sample (analyte), a buffer solution, a polymerase, and a primer solution are added into the capillary, whereby RT action and PCR needn't be respectively undertaken in different batches but can be undertaken in an identical capillary.

Another objective of the present invention is to provide an RT-PCR method, wherein a lyophilization process removes 95-99% water from an RT reagent to obtain a lyophilized RT reagent, which is completely dehydrated and lightweight, and which can be stored for long term at ambient temperature, neither denaturing nor affecting the correctness of tests, and which can be transported for long distance at ambient temperature.

A further objective of the present invention is to provide an RT-PCR method, wherein the RT reagent is fabricated into a lyophilized RT reagent with a lyophilization process and can be stored for a longer time, and wherein the lyophilized RT reagent is stored in a capillary and directly re-dissolved inside the capillary to implement RT-PCR, whereby the operating time is shortened, and the solution of reactants is exempted from contamination.

The present invention proposes an RT-PCR method comprising steps of preparing a capillary and adding an RT enzyme into a capillary; using a vacuum concentrating-freezing process to fabricate the RT enzyme into a lyophilized RT reagent; adding an RNA sample, a buffer solution, a polymerase, and a primer solution into the capillary to mix with lyophilized RT reagent and re-dissolve the lyophilized RT reagent. The RT-PCR method of the present invention enables RT and PCR to take place inside an identical capillary.

According to one embodiment of the present invention, the RT-PCR method comprises Step S1: preparing a capillary, and adding an RT enzyme into the capillary; and Step S2: undertaking a lyophilization process to fabricate the RT enzyme inside the capillary into a lyophilized RT reagent.

According to one embodiment of the present invention, the RT-PCR method further comprises Step S3: adding an RNA sample and a buffer solution into the capillary to re-dissolve the lyophilized RT reagent.

According to one embodiment of the present invention, a polymerase and a primer solution are also added to the capillary in Step S3, whereby PCR of the product of the RT reaction directly takes place, succeeding to the RT reaction of the RNA sample.

According to one embodiment of the present invention, a polymerase is added into the capillary containing the RT enzyme in Step S1, and the polymerase and the RT enzyme are jointly treated with the lyophilization process in Step S2.

According to one embodiment of the present invention, the lyophilization process includes a freezing process and a drying process.

According to one embodiment of the present invention, the capillary is placed in a refrigerator in the freezing process.

According to one embodiment of the present invention, the capillary is placed in a chiller in the freezing process.

According to one embodiment of the present invention, the capillary is pumped to a vacuum state in the drying process.

According to one embodiment of the present invention, a pre-heating process is undertaken to keep the activity of the polymerase of the lyophilized RT reagent after the lyophilization process.

According to one embodiment of the present invention, the temperature of the lyophilized RT reagent is raised in an incrementally-increasing temperature gradient to facilitate storage in the pre-heating process.

The present invention proposes an improved RT-PCR method, which not only guarantees the accuracy of tests but also increases the convenience and efficiency of experiment. Further, the method of the present invention exempts the reactants from being transferred between different test tubes and prevents the reactants from being polluted. Furthermore, the present invention exempts the reactants from denaturing in transportation and storage and prevents the enzyme from losing the activity.

### BRIEF DESCRIPTION OF THE DRAWINGS

FIG. 1 shows a flowchart of an RT-PCR method according to a first embodiment of the present invention;

FIG. 2 shows a flowchart of a lyophilization process according to one embodiment of the present invention;

FIGS. 3A-3D schematically show the operations of the RT-PCR method according to the first embodiment of the present invention;

FIG. 4 shows the results of the RT-PCR process of the present invention and the conventional RT-PCR process in the agarose electrophoresis analysis;

FIG. 5 shows a flowchart of an RT-PCR method according to a second embodiment of the present invention; and

FIG. 6 shows a flowchart of an RT-PCR method according to a third embodiment of the present invention.

### DETAILED DESCRIPTION OF THE PREFERRED EMBODIMENTS

The embodiments are described in cooperation with drawings to demonstrate the technical contents of the present invention below. However, the drawings are not drafted according to the actual dimensions but sketched with proportions, sizes, dimensional changes and displacements, which are suitable for illustration.

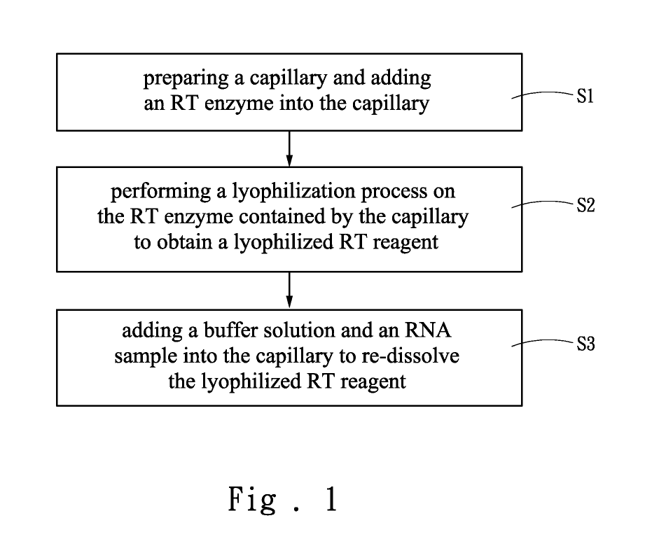

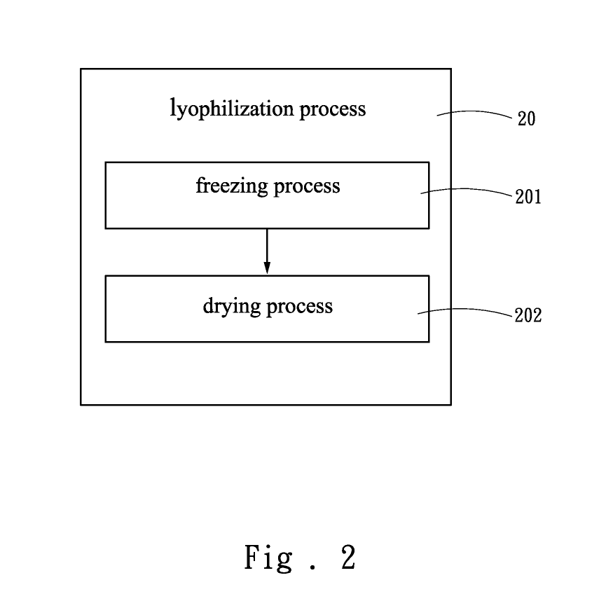

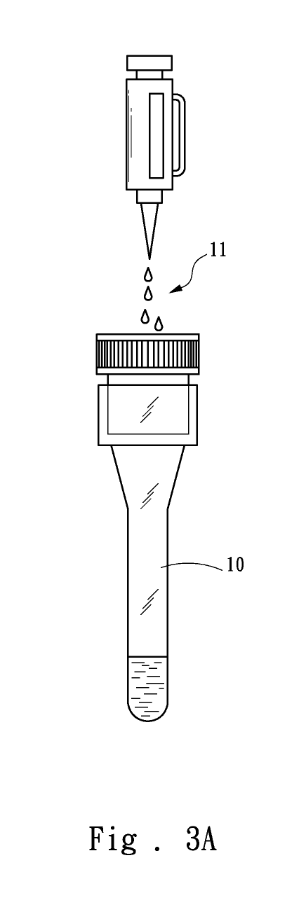

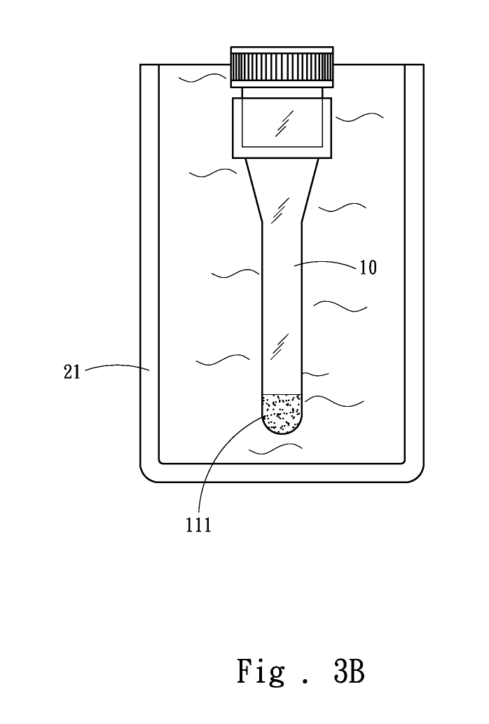

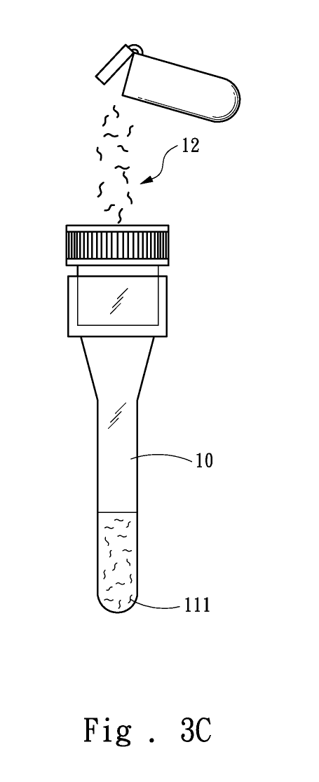

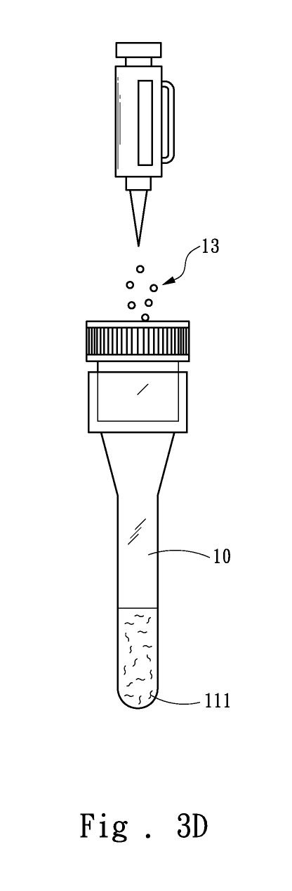

Refer to FIG. 1, FIG. 2 and FIGS. 3A-3D. According to a first embodiment of the present invention, the RT-PCR method comprises Step S1 and Step S2.

In Step S1, a capillary 10 is prepared to allow an RT enzyme 11 adding into the capillary 10, as shown in FIG. 3A.

In Step S2, a lyophilization process 20 is performed on the RT enzyme 11 inside the capillary 10. The lyophilization process 20 includes a freezing process 201 and a drying process 202. In the freezing process 201, the capillary 10 containing the RT enzyme 11 is placed in a refrigerator 21, as shown in FIG. 3B. In the drying process 202, the capillary 10 containing the RT enzyme 11 is pumped to a vacuum state. Thereby, the RT enzyme 11 inside the capillary 10 is fabricated into a lyophilized RT reagent 111, which is to facilitate RT and PCR to take place in an identical capillary latter.

The first embodiment may further comprise Step S3. In Step S3, a buffer solution (not shown in the drawings) and an RNA sample 12 are added into the capillary 10 to re-dissolve the lyophilized RT reagent 111 and enable RT and PCR to take place in the same capillary 10, as shown in FIG. 3C.

In Step S3 of the first embodiment, a polymerase 13 may be added into the capillary 10 together with an RNA sample 12, and then a primer solution (not shown in the drawings) is also added into the capillary 10, whereby PCR of the RNA sample 12 is directly undertaken after the RT reaction thereof, wherefore RT and PCR take place the same capillary 10.

Hereinbefore has been introduced the principle of the RT-PCR method of the present invention. Below is described the detailed operations of the present invention.

Refer to FIG. 2 for a flowchart of a lyophilization process according to one embodiment of the present invention. The lyophilization process 20 includes a freezing process 201 and a drying process 202. The freezing process 201 further includes a primary freezing (sublimation freezing) process and a secondary freezing (desorption freezing) process. In the primary freezing process, the RT enzyme 11 contained in the capillary 10 is placed in a refrigerator 21 at a temperature of between −20 and −40° C. for 2-4 hours to crystallize the RT enzyme 11 and sublime the redundant water. In the secondary freezing process, the crystallized RT enzyme 11 is placed in a chiller (not shown in the drawings) at a temperature of between −40 and −60° C. for 2-4 hours to pre-dry the RT enzyme 11. Then is undertaken the drying process 202. In the drying process 202, the RT enzyme 11 is dehydrated with a vacuum process at a vacuum level of less than 100 mTorr. Thus, the RT enzyme 11 is fabricated into the lyophilized RT reagent 111.

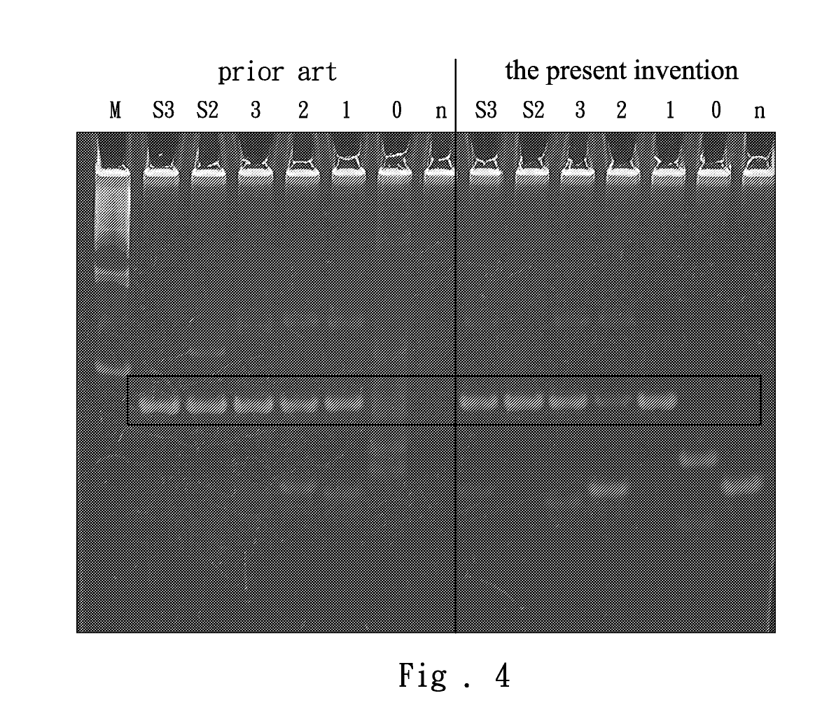

Refer to FIG. 4 for the results of the RT-PCR experiments. In FIG. 4, the result of the RT-PCR process using the lyophilized RT reagent 111 of the present invention is compared with the result of the conventional RT-PCR process.

The lyophilized RT reagent 111 is stored at a temperature of 37° C. for a week. When the lyophilized RT reagent 111 is to be used, it is re-dissolved with a re-dissolution buffer solution, sterile water, or an appropriate dilution agent. Next, 2.5% glycerol is added to the lyophilized RT reagent 111, functioning as a swelling agent. Next, the RNA sample 12, the polymerase 13 and the primer solution are added into the capillary 10 to enable PCR. In FIG. 4, a black line separates the result of the RT-PCR process of the present invention and the result of the conventional RT-PCR process. The numerals 0-3 respectively denote test results of four samples taken from the aquatic animals suspected to be infected by the necrosis virus. S2 and S3 respectively denote the standard plasmids amplified to different amounts. “n” denotes the negative contrast of the nucleic acid of normal aquatic animals. M denotes the size of molecule.

FIG. 4 shows that no obvious difference exists between the results of the RT-PCR process using the lyophilized RT reagent 111 and the conventional RT-PCR process in an agarose electrophoresis analysis. However, the RT-PCR method of the present invention outperforms the conventional RT-PCR method in convenience, efficiency and the pollution-proof performance. When RT-PCR is to be undertaken, the lyophilized RT reagent 111 can be directly re-dissolved in the capillary 10, whereby the operating time is shortened, and the risk of polluting the lyophilized RT reagent 111 is reduced.

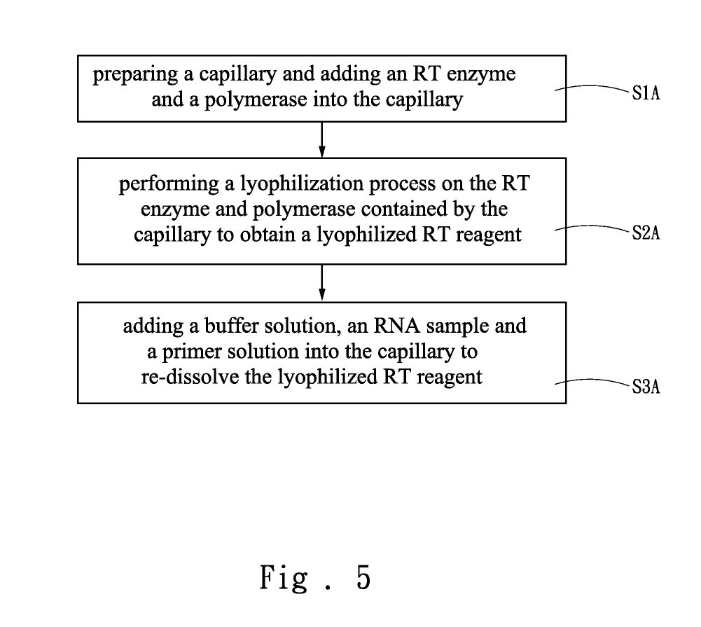

Refer to FIG. 5 for a flowchart of an RT-PCR method according to a second embodiment of the present invention. The RT-PCR method of the second embodiment comprises Step S1A, Step S2A and Step S3A, which are respectively different from Step S1, Step S2 and Step S3 of the first embodiment. In Step S1A, add the RT enzyme 11 and the polymerase 13 into the capillary 10. Step S1A is different from Step S1 in that the polymerase 13, in addition to the RT enzyme 11, is also added into the capillary 10. In Step S2A, perform the lyophilization process 20 on both the RT enzyme 11 and the polymerase 13 contained inside the capillary 10 to obtain the lyophilized RT reagent 111. Step S2A is different from Step S2 in that the RT enzyme 11 and the polymerase 13 are jointly treated by the lyophilization process 20. In Step S3A, add the buffer solution, the RNA sample 12 and the primer solution to the capillary 10 to re-dissolve the lyophilized RT reagent 111 to enable RT and PCR to take place in the same capillary 10.

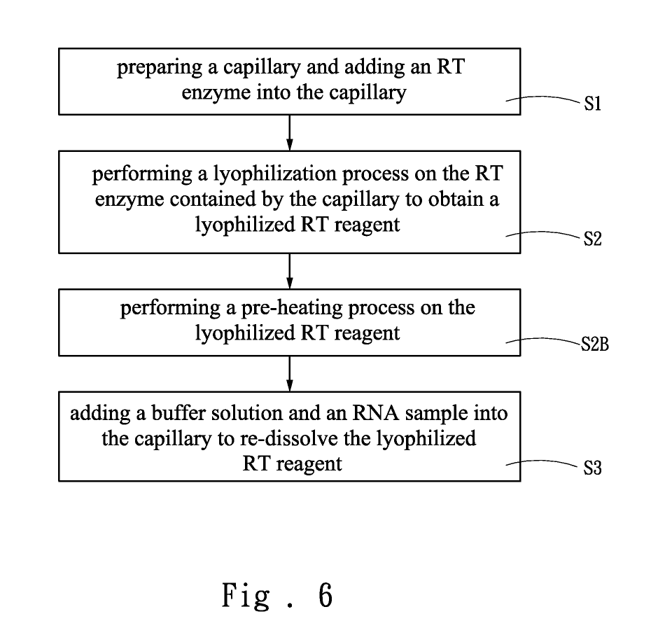

Refer to FIG. 6 for a flowchart of an RT-PCR method according to a third embodiment of the present invention. The third embodiment is different from the first embodiment in Step S2B. In Step S2B, a pre-heating process is performed on the lyophilized RT reagent 111 after the lyophilization process 20 to keep the activity of the RT enzyme 11. The pre-heating process is undertaken in an incrementally-increasing gradient of temperature, wherein the lyophilized RT reagent 111 is stored at a temperature of −10° C. for 6 hours and then stored at a temperature of 26° C. for 6 hours. Via the stepwise temperature variation, the lyophilized RT reagent 111 can be airtightly preserved at ambient temperature lest too great a temperature variation cause the lyophilized RT reagent 111 to lose its activity. Thus is promoted the accuracy of tests.

Therefore, the method of the present invention exempts the RT-PCR process from troublesome operations of adding the reactants in different batches. Further, the method of the present invention also exempts the storage and transportation of the reactants from the inconvenience of using a freezing device.

In conclusion, the present invention has the following characteristics:
1. The method of the present invention fabricates the lyophilized RT reagent 111 inside a capillary 10 and then directly undertakes the RT-PCR process in the capillary 10 and thus exempts the enzyme, polymerase and buffer solution from being added in different batches. Therefore, the method of the present invention is highly clean and less likely to be polluted by bacteria and particulates.
2. The lyophilized RT reagent 111 contained inside a capillary 10 is directly re-dissolved to implement RT-PCR. Therefore, the method of the present invention uses shorter operating time and has higher efficiency.
3. The lyophilized RT reagent 111 stored inside a capillary 10 is completely dehydrated and lightweight. Therefore, the lyophilized RT reagent 111 can be stored for a long time or transported for a long distance at ambient temperature, neither denaturing nor affecting the test results.
4. The lyophilized RT reagent 111 is processed in mild conditions and crystallized at low pressure and low temperature. Therefore, the method of the present invention exempts the RT enzyme 11 from being decomposed by high pressure or high temperature.
5. The lyophilized RT reagent 111 has very low moisture and is hard to oxidize. Therefore, the lyophilized RT reagent 111 can be stored for a long term and transported for a long distance. Further, the lyophilized RT reagent 111 can be easily rehydrated to the pre-lyophilization state.

What is claimed is:

1. A method for reverse transcription polymerase chain reaction, comprising steps:
Step S1: preparing a capillary, and adding a reverse transcription enzyme into the capillary; and
Step S2: performing a lyophilization process on the RT enzyme contained by the capillary to fabricate the RT enzyme into a lyophilized RT reagent in the capillary.

2. The method for reverse transcription polymerase chain reaction according to claim 1, further comprising step:
Step S3: adding a buffer solution and an RNA sample into the capillary to re-dissolve the lyophilized RT reagent.

3. The method for reverse transcription polymerase chain reaction according to claim 2, wherein a polymerase and a primer solution are added into the capillary in Step S3 to enable a polymerase chain reaction (PCR) of the RNA sample to directly take place inside the capillary after an RT reaction.

4. The method for reverse transcription polymerase chain reaction according to claim 1, wherein a polymerase is added into the capillary containing the RT enzyme in Step S1, and that the RT enzyme and the polymerase are jointly treated with the lyophilization process in Step S2.

5. The method for reverse transcription polymerase chain reaction according to claim 1, wherein the lyophilization process includes a freezing process and a drying process.

6. The method for reverse transcription polymerase chain reaction according to claim 5, wherein the capillary is placed in a refrigerator in the freezing process.

7. The method for reverse transcription polymerase chain reaction according to claim 5, wherein the capillary is placed in a chiller in the freezing process.

8. The method for reverse transcription polymerase chain reaction according to claim 5, wherein the capillary is pumped to a vacuum state in the drying process.

9. The method for reverse transcription polymerase chain reaction according to claim 1, wherein a pre-heating process is undertaken after the lyophilization process to keep activity of the RT enzyme of the lyophilized RT reagent.

10. The method for reverse transcription polymerase chain reaction according to claim 9, wherein the pre-heating process heats the lyophilized RT reagent in an incrementally-increasing temperature gradient to facilitate storage of the lyophilized RT reagent.
